(19) United States
(12) Patent Application Publication
(10) Pub. No.: US 2023/0125500 A1
(43) Pub. Date: Apr. 27, 2023

(54) ANGIOSTATIN NEUTRALIZING PEPTIDES DERIVED FROM ATP SYNTHASE ALPHA SUBUNIT

(71) Applicant: The Governors of the University of Alberta, Edmonton (CA)

(72) Inventors: Paul JURASZ, St. Albert (CA); Khaled Barakat, Edmonton (CA); Subha Kalyaanamoorthy, Waterloo (CA)

(21) Appl. No.: 17/937,650

(22) Filed: Oct. 3, 2022

Related U.S. Application Data
(60) Provisional application No. 63/251,133, filed on Oct. 1, 2021.

(51) Int. Cl. A61K 38/43; A61P 9/10

(52) U.S. Cl. CPC A61K 38/43; A61P 9/10

(57) ABSTRACT

Angiostatin Neutralizing Peptides Derived from ATP Synthase Alpha.

### SEQUENCE LISTING

The instant application contains a sequence listing which has been submitted electronically in XML format and is hereby incorporated by reference in its entirety. Said XML copy, created on Oct. 3, 2022, is named “PAT 109561-2 Sequence Listing ST26.xml” and is 4,376 bytes in size.

### FIELD

The present disclosure relates generally to Angiostatin Neutralizing Peptides Derived from ATP Synthase Alpha.

### BACKGROUND

Coronary artery disease (CAD) and peripheral arterial disease (PAD) are responsible for a combined 55% of Canadian cardiovascular deaths,¹ and it is estimated that up to 30% of CAD patients are not suitable for conventional therapies such as coronary artery bypass grafting or angioplasty.² Similarly, clinical options are limited for many PAD patients,³ particularly for those with co-morbidities such as diabetes wherein vascular dysfunction contributes to impede new vessel formation.^{4,5} For these patients therapeutic angiogenesis has been a sought after treatment strategy. A large number of therapeutic angiogenesis clinical trials have been initiated and focused on supplying angiogenesis promoters such as vascular endothelial growth factor (VEGF) protein, gene therapy, or endothelial progenitor cell/stem cell based-therapies.^{2,6-8} However, to date, these trials have failed to show convincingly that therapeutic angiogenesis is an effective treatment strategy.⁵ Unfortunately, a limitation of these trials has been the sole focus on delivery of pro-angiogenic molecules/cells and not on concomitant suppression of potent counteracting endogenous negative regulators of angiogenesis such as angiostatin.

### SUMMARY

In one aspect there is provide an isolated or recombinant polypeptide, comprising:

the sequence of SEQ ID NOs 1, 2, or 3,

a biologically active fragment of any of the sequence of SEQ ID NOs 1, 2, or 3, or

a variant having at least 85% sequence identity to the sequence of SEQ ID NOs 1, 2, or 3.

In one example, the variant has least 85%, 86%, 87%, 88%, 89%, 90%, 91%, 92%, 93%, 94%, 95%, 96%, 97%, 98%, or 99% sequence identity.

In one aspect there is provided an isolated or recombinant polynucleotide, comprising:

a) a nucleotide sequence encoding a polynucleotide comprising the amino acid sequence as set forth in SEQ ID NO: 1, 2, or 3,

c) a polynucleotide that hybridizes with the complementary strand of the nucleotide sequence of a);

d) a polynucleotide that differs from a) by one or more nucleotides that are substituted, deleted, and/or inserted; or

e) a derivative of a), b), c), or d).

In one example, wherein in step (d) the polynucleotide has at least 85%, 86%, 87%, 88%, 89%, 90%, 91%, 92%, 93%, 94%, 95%, 96%, 97%, 98%, or 99% sequence identity to the nucleotide sequence encoding a polynucleotide comprising the amino acid sequence as set forth in SEQ ID NO: 1, 2, or 3.

In one aspect there is provided a method of treating a subject having, or suspected of having, a disease or condition associated characterized by insufficient angiogenesis, comprising: administering to said subject an isolated or recombinant polypeptide of claim 1, wherein the isolated or recombinant polynucleotide can be translated in said cell.

In one example, wherein the disease or condition associated characterized by insufficient angiogenesis is CAD or PAD.

In one example, wherein the disease or condition associated characterized by insufficient angiogenesis is diabetes, a pre-diabetic condition, nephropathy, retinopathy, coronary artery disease, peripheral vascular disease and associated ulcers, gangrene, pain, autonomic dysfunction, arteriosclerosis, ischemic vascular disease, ischemic heart disease, myocardial ischemia, myocardial infarction, heart failure, myocardial dysfunction, myocardial remodeling, cardiomyopathies, atherosclerotic cardiovascular disease, left main coronary artery disease, arterial occlusive disease, peripheral ischemia, peripheral vascular disease, vascular disease of the kidney, peripheral arterial disease, limb ischemia, critical leg ischemia, lower extremity ischemia, cerebral ischemia, cerebrovascular disease, retinopathy, retinal repair, remodeling disorder, von Hippel-Lindau syndrome, diabetes, hereditary hemorrhagic telengiectasia, ischemic vascular disease, Buerger's disease, stroke, renovascular disease, and ischemia associated with neurodegenerative disease such as Parkinson's and Alzheimer's disease, an ulcer, a burn, male pattern baldness, atherosclerosis, ischemic heart tissue, ischemic peripheral tissue, myocardial or cerebral infarction, or vascular occlusion or stenosis

In one example, wherein the subject is a human.

### BRIEF DESCRIPTION OF THE FIGURES

Embodiments of the present disclosure will now be described, by way of example only, with reference to the attached Figures.

FIG. 1 immunoprecipitation of angiostatin K1-4 following incubation with trypsinized recombinant F₁F₀ATP synthase subunit α resulted in identification of three a peptides which bound angiostatin.

FIG. 2A representative immunoblots demonstrating the effects of alpha peptides 1 through 3 on eNOS and MT1-MMP protein expression following incubation with angiostatin. A peptide derived from ATP synthase δ subunit was used as a non-angiostatin binding control.

FIG. 2B Summary immunoblot data demonstrating the effects of alpha peptides ‘1 through 3 on eNOS protein expression following incubation with angiostatin. N=5 experiments. Combined used alpha peptides 1-3 appears to be most effective at reversing the angiostatin inhibitory effects on eNOS protein expression. *, P<0.05 vs. Angiostatin.

FIG. 2C Summary immunoblot data demonstrating the effects of alpha peptides ‘1 through 3 on eNOS protein expression following incubation with angiostatin. N=5 experiments. Combined used alpha peptides 1-3 appears to be most effective at reversing the angiostatin inhibitory effects on MT1-MMP protein expression. *, P<0.05 vs. Angiostatin.

FIG. 3 representative gelatin zymography and summary data demonstrating the effects of alpha peptides 1 through 3 on MMP-2 protein expression following incubation with angiostatin. N=experiments. Combined used of alpha peptides 1-3 appears to be most effective at reversing the angiostatin inhibitory effects on MMP-2 protein expression. *, P<0.05 vs. Angiostatin.

FIG. 4 summary data demonstrating the effects of alpha peptides 1 through 3 on human microvascular endothelial cell migration following incubation with angiostatin. N=2 experiments. Combined used of alpha peptides 1-3 appears to be most effective at reversing the angiostatin inhibitory effects on endothelial cell migration, as early stage of angiogenesis, during hypoxia.

FIGS. 5A-5D 3 selected poses of alpha peptide 1 (TGTAEMSSILEER) (A) demonstrate likely binding of kringle 2 of angiostatin K1-3 (B-D) identification of corresponding angiostatin amino acids for peptide binding based on 3 representative poses.

FIGS. 6A-6D 3 selected poses of alpha peptide 2 (TSIAIDTIINQK) (A) demonstrate likely binding in kringles 1 and 2 of angiostatin K1-3 (B-D) identified of corresponding angiostatin amino acids for peptide binding based on 3 representative poses.

FIGS. 7A-7D 3 selected poses of alpha peptide 3 (AVDSLVPIGR) (A) demonstrate likely binding in kringles 1 and 2 of angiostatin K1-3 (B-D) identification of corresponding angiostatin amino acids for peptide binding based on 3 representative poses.

FIG. 8 Angiostatin neutralizing peptides do no inhibit fibrinolysis unlike positive control aprotinin. N=4 experiments. Thrombin 1 U/ml. Tissue plasminogen activator (tPA) 1 μg/ml. *, P<0.05 vs thrombin+tPA.

FIG. 9 summary data demonstrating the effects of alpha peptides 1 through 3 on human microvascular endothelial cell migration following incubation with angiostatin. N=4 experiments. Combined used of alpha peptides 1-3 reverses the angiostatin inhibitory effects on endothelial cell migration, as early stage of angiogenesis, during hypoxia. *, P<0.05 vs. Control. #, P<0.05 vs. Angiostatin.

FIG. 10 summary data demonstrating the effects of alpha peptides 1 through 3 on human microvascular endothelial cell nitric oxide (NO) production. N=3 experiments. Combined used of alpha peptides 1-3 reverses the angiostatin inhibitory effects on endothelial cell NO production. *, P<0.05 vs. Control. #, P<0.05 vs. Angiostatin.

FIG. 11 summary data demonstrating the effects of alpha peptides 1 through 3 on hind limb perfusion ratio (I/NI) over 28 days. I—ischemic limb, NI—non-ischemic limb. N=9 mice in each group. ***, P<0.001 vs. delta peptide.

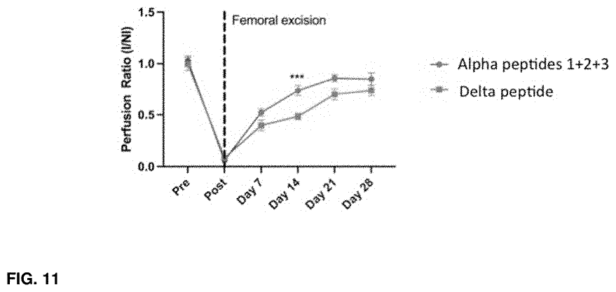

### DETAILED DESCRIPTION

Generally, the present disclosure provides Angiostatin Neutralizing Peptides Derived from ATP Synthase Alpha.

This is a new use for peptides derived from the recombinant protein of human ATP synthase subunit alpha. We have found that these peptides inhibit the function of the potent endogenous angiogenesis inhibitor angiostatin. Therefore, it has the potential to be utilized as a novel therapeutic promoting new blood vessel growth.

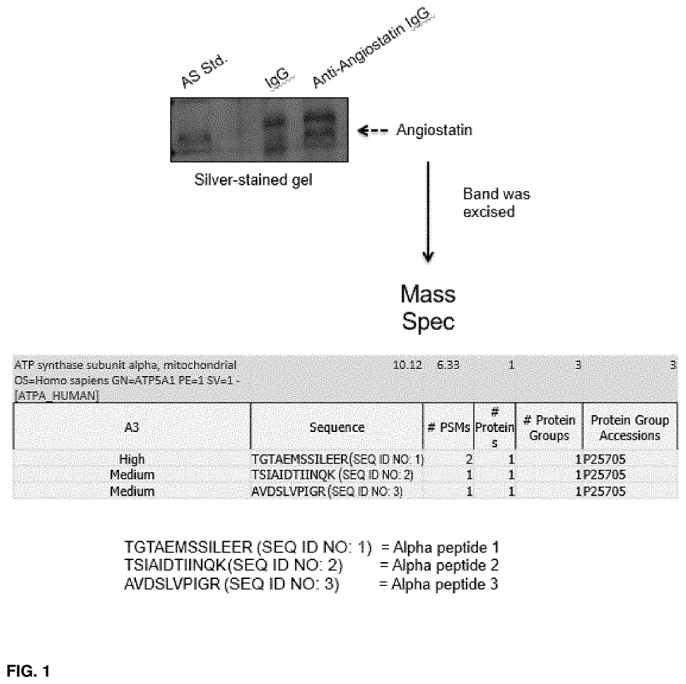

Angiostatin was first discovered in a Lewis Lung carcinoma model of concomitant resistance.⁹ It is a cleavage product of plasminogen (FIG. 1), a liver-synthesized plasma beta-globulin responsible for fibrinolysis.¹⁰ The originally described angiostatin contained the first four of five plasminogen subunits named kringles (angiostatin K1-4),¹¹ although other angiostatins with differing kringle numbers and angiostatic activity have also been discovered including angiostatin K1-3 which may be found in blood at lower concentrations compared to angiostatin K1-4.¹¹ We have found that angiostatin K1-4 (hereto referred as angiostatin) is constitutively generated by circulating platelets via a proteolytic mechanism on their membrane surface resulting in plasma concentrations of ˜30 μg/ml (600 nM), and it is the predominant isoform found in plasma. Platelet-generated angiostatin is also taken up into and stored in platelet α-granules, and further secreted upon aggregation. At physiological concentrations and normoxic conditions, it has relatively benign effects on endothelial cell viability and/or migration. However, under hypoxic conditions as would be present in CAD or PAD, angiostatin potently suppresses endothelial nitric oxide synthase (eNOS) and endothelial matrix metalloproteinase-2 and -14 (MMP-2 and -14) expression thus impairing endothelial cell migration, a critical early step for successful angiogenesis. Hence selective angiostatin neutralization may enhance hypoxia-induced angiogenesis resulting in improved oxygen and nutrient delivery to hypoxic tissues of patients with coronary artery and peripheral artery disease.

To block angiostatin action and improve angiogenesis a number of strategies may be pursued I) antibody-mediated angiostatin neutralization and ii) aprotinin-mediated inhibition of angiostatin generation by platelets.¹²⁻¹⁴ However, important potential obstacles exist to using these two strategies clinically. First, as angiostatin is a cleavage product of plasminogen, to date, all angiostatin-binding antibodies also cross-react with plasminogen, which is more abundant in plasma (˜2 μM).^{12,15} Hence, a neutralizing antibody could potentially be “mopped” up by plasminogen. Second, in addition to inhibiting angiostatin formation by platelets, aprotinin inhibits plasminogen/plasmin activity potentially promoting pathological thrombosis by inhibiting clot breakdown. Hence, a need exists to develop novel selective angiostatin inhibitors that also do not impair fibrinolysis for therapeutic angiogenesis promotion. In our report of invention we demonstrate that the combined use of three peptides derived from the F₁ ATP synthase a subunit, a known angiostatin but not plasminogen binding partner,¹⁶ reverses the inhibitory effects of angiostatin on endothelial cell eNOS, MMP-2, and MMP-14 protein expression and increases endothelial cell migration; thus potentially promoting an early stage of angiogenesis. Further, we demonstrate these three peptides do not inhibit fibrinolysis in an in vitro assay. And, we also demonstrate via molecular modeling that the three peptides also likely bind angiostatin k1-3, with probable binding sites for the three peptides within kringle 1 and 2. However, kringle 2 appears to be the more likely binding site with angiostatin amino acids arginine234, tryptophan235, tyrosine 200, arginine220, glutamic acid 221 and tryptophan 225 forming the binding site within the kringle 2 domain

Therapeutic angiogenesis (growth of new blood vessels) has been a long sought after treatment strategy for coronary artery disease (CAD) and peripheral arterial disease (PAD) patients not suitable for conventional therapies such as coronary artery bypass grafting or angioplasty. A large number of therapeutic angiogenesis clinical trials have been initiated and focused on supplying angiogenesis promoters such as vascular endothelial growth factor (VEGF) or basic fibroblast growth factor (bFGF) protein, gene therapy, or endothelial progenitor cell/stem cell based-therapies. However, to date, these trials have failed to show convincingly that therapeutic angiogenesis is an effective treatment strategy. Unfortunately, a limitation of these trials has been the sole focus on delivery of pro-angiogenic molecules/cells and not on concomitant suppression of counteracting endogenous negative regulators of angiogenesis, such as the potent angiogenesis inhibitor angiostatin.

Our angiostatin binding peptides reverse the angiogenesis inhibiting effects of angiostatin on endothelial cells, which is normally made by human platelets and found in blood, thereby potentially promoting growth of new blood vessels by allowing endogenous pro-angiogenic molecules normally found within blood to work. Importantly, angiostatin is a hypoxia-specific angiogenesis inhibitor; therefore, angiogenesis would be expected to promote new blood vessel growth only in hypoxic/ischemic tissues such as those found in CAD and PAD and potentially would have minimal side effects in other healthy tissues. Furthermore, these angiostatin binding peptides does not inhibit angiostatin's parent molecule, plasminogen/plasmin, thereby they do not interfere with fibrinolysis.

In some aspects there is provided herein polypeptides herein may be used to induce angiogenesis in a subject.

Thus, according to one aspect, there is provided methods of inducing angiogenesis in a subject in need thereof.

The term “subject”, as used herein, refers to an animal, and may include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. The subject may be an infant, a child, an adult, or elderly. In a specific example, the subject is a human.

As used herein, the term “angiogenesis” refers to the expansion of current blood vessels or the creation of new blood vessels. “Angiogenesis” may also refers to the expansion of current blood vessels or the creation of new blood vessels above normal/regular vascular repair.

In some examples, a subject is one in need of increased angiogenesis.

In one example, the subject in need of increased angiogenesis has coronary artery disease (CAD), is at risk of developing CAD, or is suspected of having CAD.

In some examples, “coronary artery disease” refers to a condition in which plaque builds up inside the coronary arteries. The build up of plaque occurs over many years. Over time, plaque hardens and narrows the coronary arteries. This limits the flow of oxygen-rich blood to the heart muscle. If the flow of oxygen-rich blood to the heart muscle is reduced or blocked, angina or heart attack may occur

In one example, the subject in need of increased angiogenesis has peripheral arterial disease (PAD), is at risk of developing PAD, or is suspected of having PAD.

In some examples, “peripheral arterial disease” refers to a narrowing of the blood vessels outside of the heart. This happens when plaque, a substance made up of fat and cholesterol, builds up on the walls of the arteries that supply blood to the arms, pelvis and legs. The plaque causes the arteries to narrow or become blocked. This can reduce or stop blood flow, usually to the legs, causing them to hurt or feel numb. If severe enough, blocked blood flow can cause tissue death.

It will be appreciated that the polypeptides as described herein may be used to induce angiogenesis in a subject.

In some aspects, there is provided polypeptides and polynucleotides which may be used to induce angiogenesis.

In some aspects, there is provided polypeptides and polynucleotides which may be used to induce angiogenesis and treat a disease or condition characterized by insufficient angiogenesis.

Thus, in some aspects there is provided polypeptides and polynucleotides which may be used to induce angiogenesis and treat a disease or condition characterized by insufficient angiogenesis.

In a specific example, a disease or condition characterized by insufficient angiogenesis is CAD.

In a specific example, a disease or condition characterized by insufficient angiogenesis is PAD.

In other specific examples, a disease or condition characterized by insufficient angiogenesis is diabetes (for example, type I or type II), a pre-diabetic condition, nephropathy, retinopathy, coronary artery disease, peripheral vascular disease and associated ulcers, gangrene, and/or pain, and/or autonomic dysfunction, arteriosclerosis, ischemic vascular disease, ischemic heart disease, myocardial ischemia, myocardial infarction, heart failure, myocardial dysfunction, myocardial remodeling, cardiomyopathies,), atherosclerotic cardiovascular disease, left main coronary artery disease, arterial occlusive disease, peripheral ischemia, peripheral vascular disease, vascular disease of the kidney, peripheral arterial disease, limb ischemia, critical leg ischemia, lower extremity ischemia, cerebral ischemia, cerebrovascular disease, retinopathy, retinal repair, remodeling disorder, von Hippel-Lindau syndrome, diabetes, hereditary hemorrhagic telengiectasia, ischemic vascular disease, Buerger's disease, and ischemia associated with neurodegenerative disease such as Parkinson's and Alzheimer's disease.

In other examples, a disease or condition characterized by insufficient angiogenesis is a wound, an anastomosis, an ulcer, a burn, male pattern baldness, atherosclerosis, ischemic heart tissue, ischemic peripheral tissue (for example, limb or mesentery ischemia), myocardial or cerebral infarction, or vascular occlusion or stenosis.

As used herein the phrase “a subject in need thereof” refers to a subject who is diagnosed with, predisposed to or suspect of having or at risk of developing a disease or condition characterized by insufficient angiogenesis.

The terms “polypeptide” and “protein” are used interchangeably herein and refer to a molecular chain of two or more amino acids linked through peptide bonds. The terms do not refer to a specific length of the product. Thus, “peptides”, “oligopeptides”, and “protein” are included within the definition of polypeptide.

As used herein, a “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence.

In some examples, the polypeptide is post-translationally modified. Post-translational modification of the polypeptide refers to, for example, glycosylations, acetylations, phosphorylations and the like.

In some examples, the polypeptide includes one or more amino acid analogs or non-canonical or unnatural amino acids are included as can be synthesized, or expressed recombinantly using known protein engineering techniques. In addition, polypeptides may be derivatized as by well-known organic chemistry techniques.

As used herein, the term “nucleic acid” or “polynucleotide” refers to genomic DNA (gDNA), complementary DNA (cDNA) or DNA. Polynucleotides include single and double stranded polynucleotides, either recombinant, synthetic, or isolated. In some exampes, polynucleotide refers to messenger RNA (mRNA), RNA, genomic RNA (gRNA), plus strand RNA (RNA(+)), minus strand RNA (RNA(−)).

As used here, “polynucleotide variant” refers to molecules which differ in their nucleic sequence from a native or reference sequence. The nucleic acid sequence variants may possess substitutions, deletions, and/or insertions at certain positions within the nucleic acid sequence, as compared to a native or reference sequence.

As used herein, the term “derivative” is used synonymously with the term “variant” and refers to a molecule that has been modified or changed in any way relative to a reference molecule or starting molecule.

In some examples, polynucleotides include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein, typically where the variant maintains at least one biological activity of the reference sequence.

As used herein, the terms “polynucleotide variant” and “variant” and the like refer to polynucleotides displaying substantial sequence identity with a reference polynucleotide sequence or polynucleotides that hybridize with a reference sequence under, for example, stringent conditions. These terms may include polynucleotides in which one or more nucleotides have been added or deleted, or replaced with different nucleotides compared to a reference polynucleotide. It will be understood that that certain alterations inclusive of mutations, additions, deletions and substitutions can be made to a reference polynucleotide whereby the altered polynucleotide retains the biological function or activity of the reference polynucleotide.

The polynucleotide described herein may be used in a vector.

The term “vector” as used herein refers to any nucleic acid molecule for the cloning of and/or transfer of a nucleic acid into a cell. A vector may be a replicon to which another nucleotide sequence may be attached to allow for replication of the attached nucleotide sequence.

As used herein, the term “isolated” refers to material, for example a polynucleotide, a polypeptide, or a cell, that is substantially or essentially free from components that normally accompany it in its native state.

The term “introducing” as used herein in the context of a cell or organism refers to presenting the nucleic acid molecule and/or polypeptide to the organism and/or cell in such a manner that the nucleic acid molecule and/or polypeptide gains access to the interior of a cell.

A “cell” or “host cell” refers to an individual cell or cell culture that can be or has been a recipient of any recombinant vector(s), isolated polynucleotide, or polypeptide. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a recombinant host cell.

In one example, the host cell is a cell obtained or derived from a subject.

In one example the host cell is a human cell.

As used herein, the term “biologically active fragment” refers to a fragment of a full-length parent polypeptide which fragment retains the activity of the parent polypeptide. A biologically active fragment therefore may modulate angiogenesis.

The term “functional” when used in conjunction with “derivative” or “variant” refers to a which possess a biological activity that is substantially similar to a biological activity of the entity or molecule of which it is a derivative or variant.

Polypeptides may be obtained through the application of standard recombinant nucleic acid techniques or synthesized using conventional liquid or solid phase synthesis techniques.

As used herein “corresponds to” or “corresponding to” includes a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window.

The term “hybridization” as used herein refers to the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that do not hybridise efficiently.

As used herein, the term “stringent hybridization” refers to conditions under which polynucleic acid hybrids are stable. As known to those of skill in the art, the stability of hybrids is reflected in the melting temperature (Tm) of the hybrids, which is determined by the G:C content of the sequence, sequence length, and other physical characteristics and hybridization conditions known in the art. For example, the amount of sodium ion present affects the stability of a hybrid. Typically, the hybridization or binding step is performed under conditions of lower stringency, followed by washes of varying, but higher, stringency. Reference to the level of hybridization stringency relates to such washing conditions.

As used herein, the term “high stringency hybridization” and the like refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C.

In some examples, a pharmaceutical composition is prepared in a manner known in the art, with pharmaceutically inert inorganic and/or organic excipients being used.

The term ‘pharmaceutically acceptable’ refers to molecules and compositions that are physiologically tolerable and do not typically produce an allergic or similarly untoward reaction when administered to a patient.

In some examples, the pharmaceutical composition may be formulated as a pill, tablet, coated tablet, hard gelatin capsule, soft gelatin capsule and/or suppository, solution and/or syrup, injection solution, microcapsule, implant and/or rod, and the like.

In some examples, the pharmaceutical composition may be formulated as an injection solution.

In some examples, pharmaceutically acceptable excipients for preparing pills, tablets, coated tablets and hard gelatin capsules may be selected from any of: Lactose, corn starch and/or derivatives thereof, talc, stearic acid and/or its salts, etc.

In some examples, pharmaceutically acceptable excipients for preparing soft gelatin capsules and/or suppositories may be selected from fats, waxes, semisolid and liquid polyols, natural and/or hardened oils, etc.

In some examples, pharmaceutically acceptable excipients for preparing solutions and/or syrups may be selected from water, sucrose, invert sugar, glucose, polyols, etc.

In some examples, pharmaceutically acceptable excipients for preparing injection solutions may be selected from water, saline, alcohols, glycerol, polyols, vegetable oils, etc.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

Experimental procedures:

Trypsin digestion of ATP synthase subunit α. Recombinant F₁F₀ ATP synthase subunit α was digested with trypsin, and following heat-inactivation of trypsin the resulting peptides incubated with purified angiostatin kringles 1-4. Subsequently, the angiostatin was immunoprecipitated and submitted for mass spectroscopy analysis to identify any potential bound peptides of subunit α.

Cell culture and hypoxia. Cardiac-derived Human microvascular endothelial cells from (HMVEC-C) were obtained from Lonza (Walkersville, Md., USA) and cultured in a humidified atmosphere at 37° C. and 5% CO2 in EGM-2 MV. Depending on the donor, the primary endothelial cells were serum starved for 4-16 h in EBM-2 and 0.5% FBS prior to exposure to hypoxic conditions. The time of serum starvation prior to treatment was titrated to induce approximately 40% apoptosis following treatment under normoxic conditions. Hypoxia was induced by exposing endothelial cells for 48 h under serum-starved conditions in a Billups-Rothenberg chamber continuously gassed with 95% N2-5% CO2.

Immunoblots. HMVEC-L lysates were subjected to 8% sodium dodecyl sulfate-polyacrylamide gel electrophoresis Blots were blocked overnight in TTBS and 5% non-fat milk and then cut at the 50 KDa across the membrane. Subsequently the >50 KDa upper membrane was incubated with either rabbit anti-human MMP-14 (MT1-MMP) (1 μg/ml) (Millipore) (Etobicoke, ONT, Canada), or mouse anti-eNOS (1:1000) (Abcam) (Toronto, ONT, Canada) for 2 h. Anti-mouse (1:10,000), or anti-rabbit (1:5000) horseradish peroxidase-conjugated antibodies were used as the secondary antibodies (Sigma, Oakville, ON, Canada). Immunoreactive bands were visualized with ECL Plus (Amersham Biosciences, San Francisco, Calif.). The bottom portions of the membranes (<50 KDa) were probed for β-actin with a β-actin horseradish peroxidase-conjugated antibody (1:20,000) (Sigma), which was used as a loading control. Blot bands were quantified using ImageJ software (Bio-Rad, Mississauga, ON, Canada) and expressed as arbitrary units of density per β-actin.

Gelatin zymography. Gelatin zymography was used to assess MMP-2 levels within HMVEC-C lysates. Zymography was performed using 8% SDS-PAGE with copolymerized gelatin (2 mg/ml). After electrophoresis, gels were washed 3× for 20 min in 2% Triton X-100. Next, gels were washed 2· for 20 min in developing buffer (50 mM Tris-HCl pH 7.6, 150 mM NaCl, 5 mM CaCl2), and 0.05% NaN3) and then incubated in developing buffer at 37° C. overnight. Gels were stained with 0.05% Coomassie Brilliant Blue and then destained in a 4% ethanol and 8% acetic acid solution. Gelatinolytic activity was detected as transparent bands against a blue background of Coomassie-stained gelatin. Seventy-two kilodaltons pro-MMP-2 activity was identified by comparison with molecular weight markers.

Migration assays. 24-well plate inserts with 8.0 μM pores (Falcon Corning, Tewksbury, Mass., USA) were coated with 1 mg/ml gelatin and incubated at 37° C. for 2 hours to allow gelatin to solidify. Excess gelatin was aspirated and inserts were left to dry overnight under UV light, then gently washed once with 100μl of PBS. Once approximately 80% confluent, HMVEC-C were serum starved for 8 hours in 0.5% FBS in EBM-2. Cells were trypsinized and seeded into the top chamber of the gelatin-coated inserts at a concentration of 25 000 cells/insert. Recombinant VEGF (10 ng/ml) in 0.5% FBS in EBM-2 was added to the corresponding bottom chambers to serve as a chemoattractant. HMVEC-C were treated with PBS control, angiostatin (600 nM), angiostatin (600 nM)+a peptide 1 through 3 (3 μM), angiostatin (600 nM)+negative-control 6 peptide (3 μM), and angiostatin (600 nM)+combined a peptide 1-3 (1 μM each). The system was incubated in hypoxia (95% N2, 5% CO2) for 48 hours. After incubation, the top layer of each insert was scraped gently using a Q-tip to remove the non-migrated cells. Migrated cells at the bottom of the insert were fixed in 4% formaldehyde in PBS for five minutes, followed by staining using Diffquik stain (Siemens Healthcare Diagnostics Inc., Newark, Del., USA). Stained cells from five fields of view of 24-well inserts were imaged at 10× magnification using an Olympic CKX41 microscope (Olympus America Inc., Melville, N.Y.) equipped with an Infinity 1 digital camera. Cells were counted using ImageJ software, and expressed as migration percent of PBS control.

Fibrinolysis Assays. Citrated human plasma (platelet-free) was recalcified to a final free Ca²⁺ concentration of 2.5 mM. After recalcification, plasma was incubated with a peptide 1, 2, 3, or with combined a peptides 1-3 (each 1 μM), or with negative control 8 peptide (3 μM) or with aprotinin (10 μM) as positive control for inhibiting fibrinolysis. To initiate clotting ice-cold thrombin (0.3 U/ml) was added to each well, while to initiate fibrinolysis human tissue plasminogen activator (1 μg/ml; Sigma) was added. After 2 hours incubation at 37° C. the absorbance at 405 nm was measured using a Bio-Rad iMark microplate reader and the results were expressed as percent fibrinolysis.

Molecular Modeling. The structure of each ATP synthase alpha subunit-derived peptide was predicted using the PEP-FOLD peptide fold prediction server. The five best structural folds predicted for each peptide were used for performing blind docking on the angiostatin protein. For each alpha peptide model, the top 10 binding modes of the alpha peptide—angiostatin complex were collected. The resulting 50 poses for each of the three peptides were clustered and representative poses from each cluster were used to identify the lowest energy conformations for the complex. For each representative cluster, 600 decoy conformations were generated in the Rosetta program and the conformations with the best total score and interface score (i.e., low energy conformations) were identified. Finally, a 5 ns molecular dynamics simulation was performed on these low-energy conformations and the binding free energy estimates were obtained. Interactions were studied from the complexes that had the best binding free energies.

Nitric oxide measurement assay. Following hypoxia exposure for 48 h under serum-starved conditions in a Billups-Rothenberg chamber continuously gassed with 95% N2-5% CO2, HMVEC-C were incubated with DAF-FM diacetate (5 μM). The DAF-FM was washed out 2× with 2 ml PBS, and 100 μM L-arginine in Tyrodes's buffer added to the HMVEC-C. Subsequently, DAF-FM fluorescence analyzed by flow cytometry.

Mouse hind limb ischemia model. C57BL/6 mice were anesthetized using 1.5-2% isoflurane to obtain surgical plane of anesthesia and were place on 37° C. water heating pad to maintain body temperature. Mice were administered with combined Alpha peptides 1-3 or control delta peptide via intravenous injection to achieve a total 3 μM blood concentration of peptides based on 0.08 ml blood/gm body weight. Mice were shaved of hair around the lower abdomen and hind leg areas. A 1 cm incision was made distal of the right knee joint, and a small section of the femoral artery between the superficial epigastric artery and popliteal bifurication tied with sutures and the artery excised between suture ties. The incision was closed using subcutaneous sutures. Mice were further intravenously injected with combined Alpha peptides 1-3 or control delta peptides to achieve 3 μM blood concentrations on days 3, 7, and 11 post surgery. Laser Doppler scans were performed pre-operatively, post-operatively, and at days 7, 14, 21, and 28.

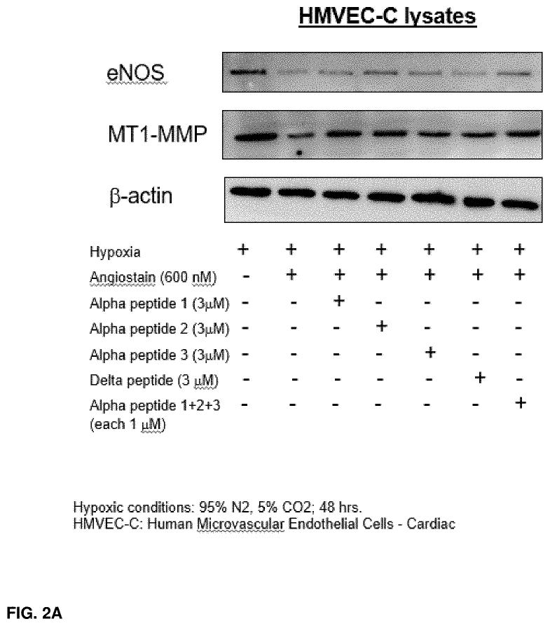

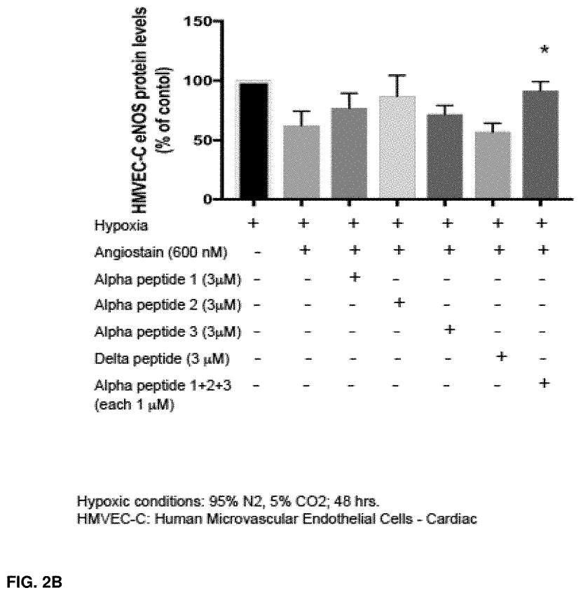

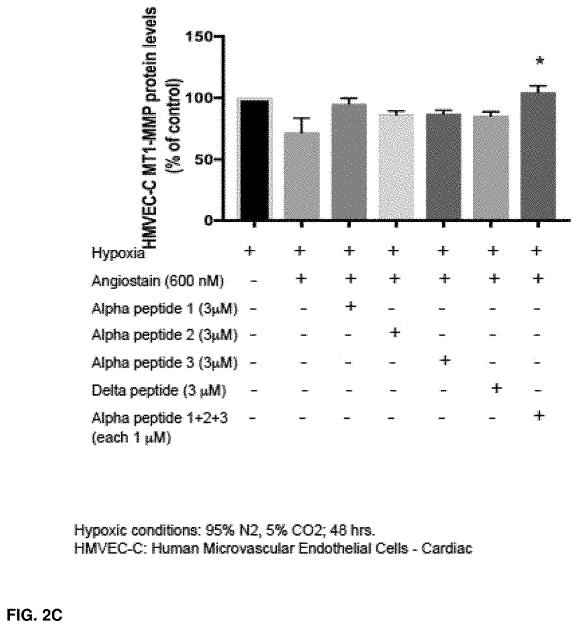

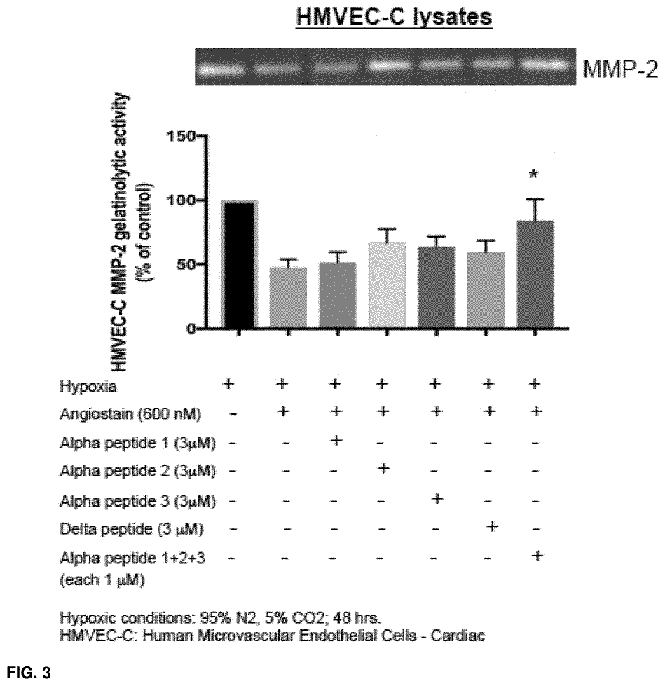

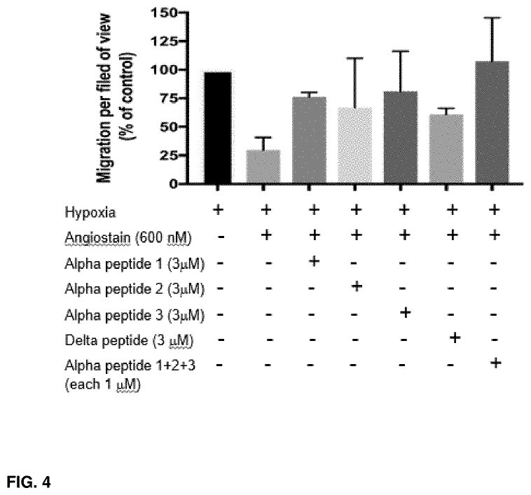

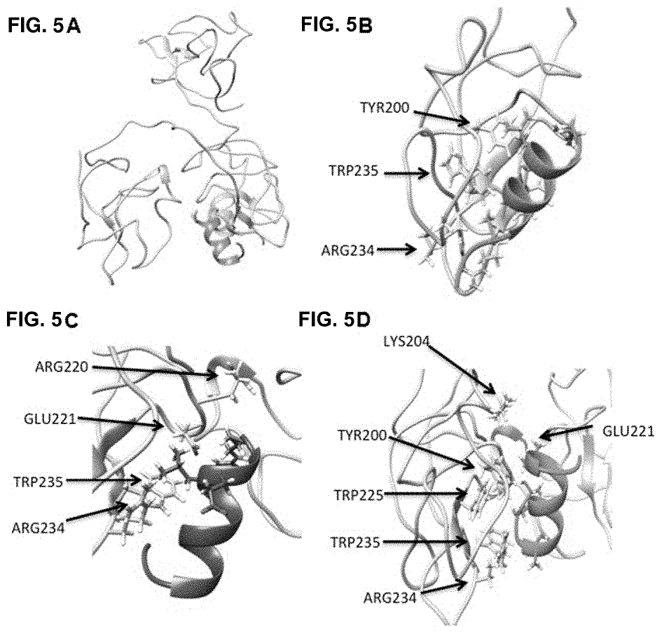

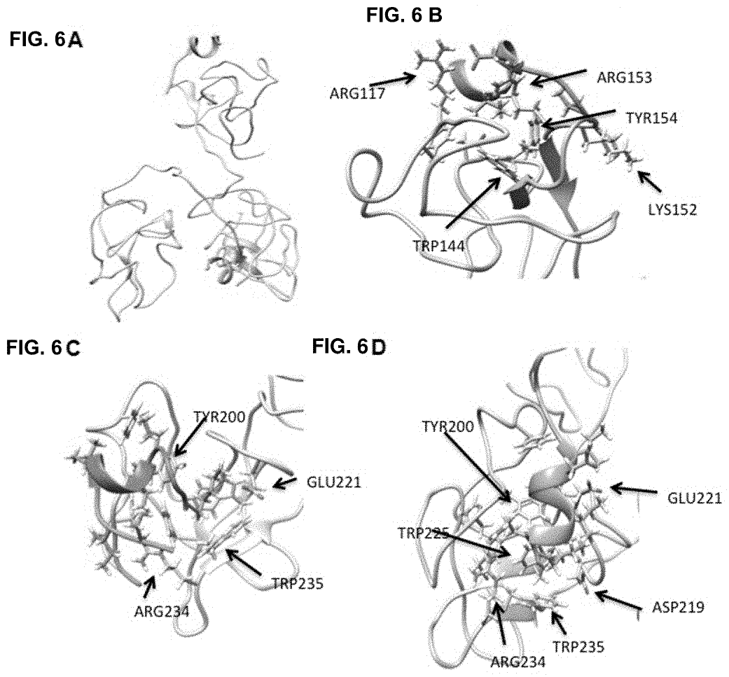

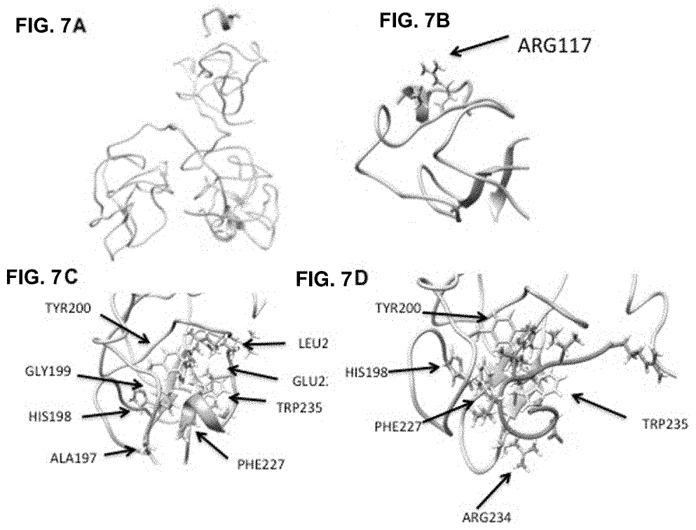

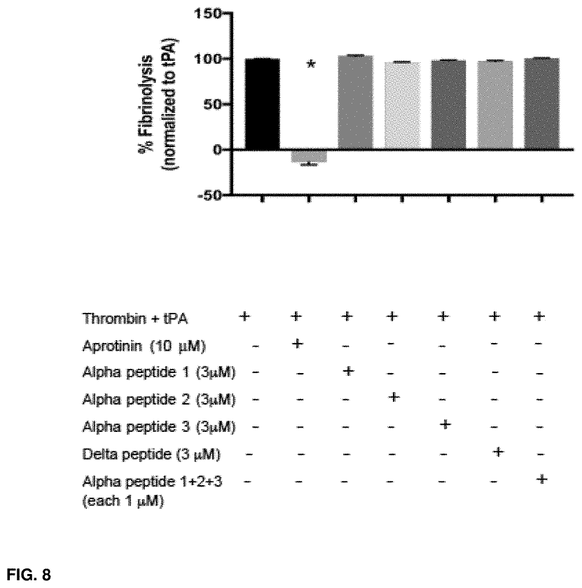

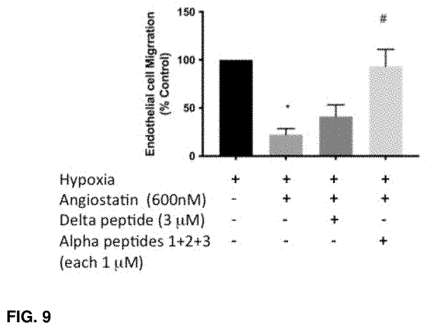

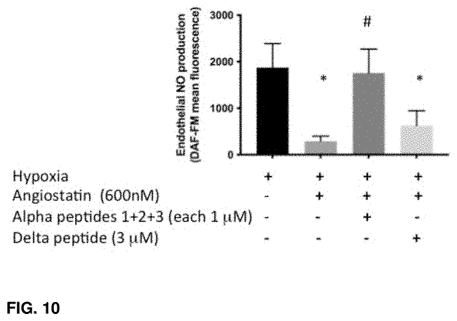

Description Following immunoprecipitation peptides derived from trypsinization of the F₁F₀ ATP synthase subunit alpha, mass spectrometry identified three ATP synthase alpha subunit peptides that bound to angiostatin (FIG. 1). The three peptides identified peptides TGTAEMSSILEER (SEQ ID NO: 1), TSIAIDTIINQK (SEQ ID NO:,2) and AVDSLVPIGR (SEQ ID NO: 3) were named as Alpha peptide 1, Alpha peptide 2, and Alpha peptide 3, respectively. Based on their peptide spectral matches (PSMs) the peptides were determined to be of high abundance for Alpha peptide 1, and medium abundance for Alpha peptides 2 and 3. In cell culture experiments with cardiac-derived human microvascular endothelial cells (HMVEC-C), treatment with angiostatin for 48 hours under hypoxic conditions resulted in a significant reduction in pro-angiogenic proteins endothelial nitric oxide synthase (eNOS) and membrane type-1 matrix metalloproteinase (MT1-MMP) as measured by immunoblot (FIGS. 2A-C) and matrix metalloproteinase-2 (MMP-2) as measured by gelatin zymography (FIG. 3). Individually, the alpha peptides failed to significantly increase eNOS, MT1-MMP, and MMP-2 levels in angiostatin-treated hypoxic HMVEC-C (FIG. 2 and FIG. 3). Similarly, a control peptide derived from the F₁F₀ ATP synthase delta subunit (VPTLQVLRPGLVV (SEQ ID NO: 4) also failed to significantly increase eNOS, MT1-MMP, and MMP-2 levels in angiostatin-treated HMVEC-C (FIG. 2 and FIG. 3). However, when combined Alpha peptides 1-3 significantly increased eNOS, MT1-MMP, and MMP-2 levels in angiostatin-treated HMVEC-C to that of non-treated controls (FIG. 2 and FIG. 3). Next, MMP-dependent endothelial migration assays were carried out with HMVEC-C as an in vitro assessment of early stage angiogenesis. Within these assays angiostatin decreased HMVEC-C migration under hypoxic conditions and the combined Alpha peptides 1-3 reversed this inhibition demonstrating angiostatin-neutralization by Alpha peptides 1-3 promotes early angiogenesis under hypoxic conditions (FIG. 4 and FIG. 9). Subsequently, in silico molecular modeling was performed to identify Alpha peptide 1-3 binding sites within angiostatin. FIG. 5A demonstrates that angiostatin kringle 2 contains the binding sites for three of the best structural folds of Alpha peptide 1. The binding site of each of these three Alpha peptide 1 folds was predicted to be composed of ARG234, TRP235, TYR200, ARG220, GLU221, TRP225 (FIG. 5B-D). FIG. 6A demonstrates that angiostatin kringle 2 contains the binding site for two of the best structural folds of Alpha peptide 2 and that angiostatin kringle 1 may also contain an Alpha 2 peptide binding site (FIG. 6-B-D). Similarly, FIG. 7A shows that angiostatin kringle 2 contains the binding site for two of the best structural folds of Alpha peptide 3 and that angiostatin kringle 1 may also contain an Alpha 3 peptide binding site (FIG. 7-B-D). In an in vitro fibrinoysis assay, Alpha peptides 1-3 did not inhibit fibrinolysis whether utilized individually or combined; unlike the known fibrinolysis inhibitor aprotinin (FIG. 8). When combined Alpha peptides 1-3 significantly increased the production of nitric oxide, an angiogenesis stimulating chemical mediator, by angiostatin-treated HMVEC-C under hypoxic conditions (FIG. 10). Finally, when combined and injected into mice, the Alpha peptides 1-3 accelerated the blood flow recovery to the ischemic hind limb compared to control delta peptide as measured by laser Doppler at day 14.

In one aspect there is provided an isolated or recombinant polypeptide, comprising:

the sequence of SEQ ID NOs 1, 2, or 3,

a biologically active fragment of any of the sequence of SEQ ID NOs 1, 2, or 3, or

a variant having at least 85% sequence identity to the sequence of SEQ ID NOs 1, 2, or 3.

In one example, the variant has least 85%, 86%, 87%, 88%, 89%, 90%, 91%, 92%, 93%, 94%, 95%, 96%, 97%, 98%, or 99% sequence identity.

In one aspect there is provided an isolated or recombinant polypeptide consisting of the sequence of SEQ ID NOs 1, 2, or 3.

In one aspect there is provided an isolated or recombinant polynucleotide, comprising:

a) a nucleotide sequence encoding a polynucleotide comprising the amino acid sequence as set forth in SEQ ID NO: 1, 2, or 3,

c) a polynucleotide that hybridizes with the complementary strand of the nucleotide sequence of a);

d) a polynucleotide that differs from a) by one or more nucleotides that are substituted, deleted, and/or inserted; or

e) a derivative of a), b), c), or d).

In one example, wherein in step (d) the polynucleotide has at least 85%, 86%, 87%, 88%, 89%, 90%, 91%, 92%, 93%, 94%, 95%, 96%, 97%, 98%, or 99% sequence identity to the nucleotide sequence encoding a polynucleotide comprising the amino acid sequence as set forth in SEQ ID NO: 1, 2, or 3.

In one example, wherein in step (c) said nucleic acid hybridizes with the complementary strand of the nucleic acid of a) under conditions of high stringency.

In one aspect there is provided a vector comprising, a recombinant polynucleotide, comprising:

a) a nucleotide sequence encoding a polynucleotide comprising the amino acid sequence as set forth in SEQ ID NO: 1, 2, or 3,

c) a polynucleotide that hybridizes with the complementary strand of the nucleotide sequence of a);

d) a polynucleotide that differs from a) by one or more nucleotides that are substituted, deleted, and/or inserted; or

e) a derivative of a), b), c), or d),

In one example, wherein in step (d) the polynucleotide has at least 85%, 86%, 87%, 88%, 89%, 90%, 91%, 92%, 93%, 94%, 95%, 96%, 97%, 98%, or 99% sequence identity to the nucleotide sequence encoding a polynucleotide comprising the amino acid sequence as set forth in SEQ ID NO: 1, 2, or 3.

In one example, wherein in step (c) said nucleic acid hybridizes with the complementary strand of the nucleic acid of a) under conditions of high stringency.

In one aspect there is provided an expression vector, a recombinant polynucleotide, comprising:

a) a nucleotide sequence encoding a polynucleotide comprising the amino acid sequence as set forth in SEQ ID NO: 1, 2, or 3,

c) a polynucleotide that hybridizes with the complementary strand of the nucleotide sequence of a);

d) a polynucleotide that differs from a) by one or more nucleotides that are substituted, deleted, and/or inserted; or

e) a derivative of a), b), c), or d),

wherein said polynucleotide is operably linked to a regulatory polynucleotide.

In one example, wherein in step (d) the polynucleotide has at least 85%, 86%, 87%, 88%, 89%, 90%, 91%, 92%, 93%, 94%, 95%, 96%, 97%, 98%, or 99% sequence identity to the nucleotide sequence encoding a polynucleotide comprising the amino acid sequence as set forth in SEQ ID NO: 1, 2, or 3.

In one example, wherein in step (c) said nucleic acid hybridizes with the complementary strand of the nucleic acid of a) under conditions of high stringency.

In one aspect there is provided a host cell comprising a recombinant polynucleotide, comprising:

the sequence of SEQ ID NOs 1, 2, or 3,

a biologically active fragment of any of the sequence of SEQ ID NOs 1, 2, or 3, or

a variant having at least 85% sequence identity to the sequence of SEQ ID NOs 1, 2, or 3.

In one example, wherein in step (c) the variant has at least 85%, 86%, 87%, 88%, 89%, 90%, 91%, 92%, 93%, 94%, 95%, 96%, 97%, 98%, or 99% sequence identity.

In one example, wherein in step (c) said nucleic acid hybridizes with the complementary strand of the nucleic acid of a) under conditions of high stringency.

In one aspect there is provided a host cell comprising an isolated or recombinant polypeptide of any one of claims 1 to 5.

In one aspect there is provided a host cell, comprising: a vector as described herein, or an expression vector as described herein.

In one example, the cell is a human cell.

In one aspect there is provided a pharmaceutical composition, comprising: an isolated or recombinant polypeptide as described herein, an isolated or recombinant polynucleotide as described herein, a vector as described herein, or an expression vector as described herein, and a pharmaceutically acceptable carrier or excipient.

In one aspect there is provided a method for modulating angiogenesis, comprising: introducing into a cell from a subject an isolated or recombinant polypeptide as described herein, an isolated or recombinant polynucleotide as described herein, a vector as described herein, an expression vector as described herein, or the pharmaceutical composition as described herein, wherein the isolated or recombinant polynucleotide can be translated in said cell.

In one aspect there is provided a method for modulating angiogenesis in a subject, comprising: administering to said subject an isolated or recombinant polypeptide as described herein, an isolated or recombinant polynucleotide as described herein, a vector as described herein, an expression vector as described herein, or the pharmaceutical composition as described herein, wherein the isolated or recombinant polynucleotide can be translated in said cell.

In one aspect there is provided a method of treating a subject having, or suspected of having, a disease or condition associated characterized by insufficient angiogenesis, comprising: administering to said subject an isolated or recombinant polypeptide as described herein, an isolated or recombinant polynucleotide as described herein, a vector as described herein, an expression vector as described herein, or the pharmaceutical composition as described herein, wherein the isolated or recombinant polynucleotide can be translated in said cell.

In one aspect there is provided a method of increasing, promoting, or stimulating growth, proliferation, or migration of a cell associated with angiogenesis, comprising: introducing into a cell an isolated or recombinant polypeptide as described herein, an isolated or recombinant polynucleotide as described herein, a vector as described herein, an expression vector as described herein, or the pharmaceutical composition as described herein, wherein the isolated or recombinant polynucleotide can be translated in said cell.

In one example, wherein the disease or condition associated characterized by insufficient angiogenesis is CAD or PAD.

In one example, wherein the disease or condition associated characterized by insufficient angiogenesis is diabetes, a pre-diabetic condition, nephropathy, retinopathy, coronary artery disease, peripheral vascular disease and associated ulcers, gangrene, pain, autonomic dysfunction, arteriosclerosis, ischemic vascular disease, ischemic heart disease, myocardial ischemia, myocardial infarction, heart failure, myocardial dysfunction, myocardial remodeling, cardiomyopathies, atherosclerotic cardiovascular disease, left main coronary artery disease, arterial occlusive disease, peripheral ischemia, peripheral vascular disease, vascular disease of the kidney, peripheral arterial disease, limb ischemia, critical leg ischemia, lower extremity ischemia, cerebral ischemia, cerebrovascular disease, retinopathy, retinal repair, remodeling disorder, von Hippel-Lindau syndrome, diabetes, hereditary hemorrhagic telengiectasia, ischemic vascular disease, Buerger's disease, and ischemia associated with neurodegenerative disease such as Parkinson's and Alzheimer's disease, an ulcer, a burn, male pattern baldness, atherosclerosis, ischemic heart tissue, ischemic peripheral tissue, myocardial or cerebral infarction, or vascular occlusion or stenosis

In one example, wherein the subject is a human.

In one aspect there is provided a kit, comprising: an isolated or recombinant polypeptide as described herein, an isolated or recombinant polynucleotide as described herein, a vector as described herein, an expression vector as described herein, or the pharmaceutical composition as described herein, and a container, and optionally instructions for the use thereof.

### REFERENCES

1. Canada. S. Deaths, by cause, Chapter IX: Diseases of the circulatory system (100 to 199), age group and sex, Canada, annual (number), CANSIM (database). 2011; Table 102-0529.
2. Lassaletta A, Chu L and Sellke F. Therapeutic neovascularization for coronary disease: current state and future prospects. Basic Research in Cardiology. 2011; 106:897-909.
3. Raval Z and Losordo D W. Cell Therapy of Peripheral Arterial Disease: From Experimental Findings to Clinical Trials. Circulation Research. 2013; 112:1288-1302.
4. Howangyin K Y and Silvestre J-S. Diabetes Mellitus and Ischemic Diseases: Molecular Mechanisms of Vascular Repair Dysfunction. Arteriosclerosis, Thrombosis, and Vascular Biology. 2014; 34:1126-1135.
5. Cooke J P and Losordo D W. Modulating the Vascular Response to Limb Ischemia: Angiogenic and Cell Therapies. Circulation Research. 2015; 116:1561-1578.
6. Kaminsky S M R T, Rosenberg J, Chiuchiolo M J, Van de Graaf B, Sondhi D, Crystal R G. Gene Therapy to Stimulate Angiogenesis to Treat Diffuse Coronary Artery Disease. Human Gene Therapy. 2013; 24:948-963.
7. Silvestre J-S, Smadja D M and Levy B I. Postischemic Revascularization: From Cellular and Molecular Mechanisms to Clinical Applications; 2013.
8. Stewart D J, Kutryk M J B, Fitchett D, Freeman M, Camack N, Su Y, Siega A D, Bilodeau L, Burton J R, Proulx G and Radhakrishnan S. VEGF Gene Therapy Fails to Improve Perfusion of Ischemic Myocardium in Patients With Advanced Coronary Disease: Results of the NORTHERN Trial. Mol Ther. 2009; 17:1109-1115.
9. O'Reilly M S, Holmgren, L., Shing, Y., Chen, C., Rosenthal, R. A., Moses, M., Lane, W. S., Cao, Y., Sage, E. H., and Folkman, J. Angiostatin: a novel angiogenesis inhibitor that mediates the suppression of metastases by a Lewis lung carcinoma. Cell. 1994; 79:315-328.
10. Lijnen H R. Elements of the Fibrinolytic System. Annals of the New York Academy of Sciences. 2001; 936:226-236.
11. G A S. Angiostatin and angiostatin-related proteins. Cancer Metast Reviews. 2000; 19:97-107.
12. Jurasz P, Alonso D, Castro-Blanco S, Murad F and Radomski M W. Generation and role of angiostatin in human platelets. Blood. 2003; 102:3217-3223.
13. Jurasz P. S-M, M. J., Radomska, A., Radomski, M. W. Generation of platelet angiostatin mediated by urokinase plasminogen activator: effects on angiogenesis. Journal of Thrombosis and Haemostasis. 2006; 4:1095-1106.
14. Radziwon-Balicka A MdIRC, Zielnik B, Doroszko A, Jurasz P.

Temporal and Pharmacological Characterization of Angiostatin Release and Generation by Human Platelets: Implications for Endothelial Cell Migration. PLoS ONE. 2013; 8:e59281.
15. Gately S, Twardowski P, Stack M S, Patrick M, Boggio L, Cundiff D L, Schnaper H W, Madison L, Volpert O, Bouck N, Enghild J, Kwaan H C and Soff G A. Human Prostate Carcinoma Cells Express Enzymatic Activity That Converts Human Plasminogen to the Angiogenesis Inhibitor, Angiostatin. Cancer Res. 1996; 56:4887-4890.
16. Moser T L, Kenan D J, Ashley T A, Roy J A, Goodman M D, Misra U K, Cheek D J and Pizzo S V. Endothelial cell surface F1-FO ATP synthase is active in ATP synthesis and is inhibited by angiostatin. Proceedings of the National Academy of Sciences. 2001; 98:6656-6661.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. An isolated or recombinant polypeptide, comprising:
a) the sequence of SEQ ID NOs 1, 2, or 3,
b) a biologically active fragment of any of the sequence of SEQ ID NOs 1, 2, or 3, or
c) a variant having at least 85% sequence identity to the sequence of SEQ ID NOs 1, 2, or 3.

2. The isolated or recombinant polypeptide of claim 1, wherein the variant has least 85%, 86%, 87%, 88%, 89%, 90%, 91%, 92%, 93%, 94%, 95%, 96%, 97%, 98%, or 99% sequence identity.

3. An isolated or recombinant polynucleotide, comprising:
a) a nucleotide sequence encoding a polynucleotide comprising the amino acid sequence as set forth in SEQ ID NO: 1, 2, or 3,
c) a polynucleotide that hybridizes with the complementary strand of the nucleotide sequence of a);
d) a polynucleotide that differs from a) by one or more nucleotides that are substituted, deleted, and/or inserted; or
e) a derivative of a), b), c), or d).

4. The isolated or recombinant polynucleotide of claim 3, wherein in (d) the polynucleotide has at least 85%, 86%, 87%, 88%, 89%, 90%, 91%, 92%, 93%, 94%, 95%, 96%, 97%, 98%, or 99% sequence identity to the nucleotide sequence encoding a polynucleotide comprising the amino acid sequence as set forth in SEQ ID NO: 1, 2, or 3.

5. A method of treating a subject having, or suspected of having, a disease or condition associated characterized by insufficient angiogenesis, comprising: administering to said subject an isolated or recombinant polypeptide of claim 1, wherein the isolated or recombinant polynucleotide can be translated in said cell.

6. The method of claim 5, wherein the disease or condition associated characterized by insufficient angiogenesis is CAD or PAD.

7. The method of claim 6, wherein the disease or condition associated characterized by insufficient angiogenesis is diabetes, a pre-diabetic condition, nephropathy, retinopathy, coronary artery disease, peripheral vascular disease and associated ulcers, gangrene, pain, autonomic dysfunction, arteriosclerosis, ischemic vascular disease, ischemic heart disease, myocardial ischemia, myocardial infarction, heart failure, myocardial dysfunction, myocardial remodeling, cardiomyopathies, atherosclerotic cardiovascular disease, left main coronary artery disease, arterial occlusive disease, peripheral ischemia, peripheral vascular disease, vascular disease of the kidney, peripheral arterial disease, limb ischemia, critical leg ischemia, lower extremity ischemia, cerebral ischemia, cerebrovascular disease, retinopathy, retinal repair, remodeling disorder, von Hippel-Lindau syndrome, diabetes, hereditary hemorrhagic telengiectasia, ischemic vascular disease, Buerger's disease, stroke, renovascular disease, and ischemia associated with neurodegenerative disease such as Parkinson's and Alzheimer's disease, an ulcer, a burn, male pattern baldness, atherosclerosis, ischemic heart tissue, ischemic peripheral tissue, myocardial or cerebral infarction, or vascular occlusion or stenosis

8. The method of claim 7, wherein the subject is a human.
